(19) United States
(12) Patent Application Publication
(10) Pub. No.: US 2007/0092939 A1
(43) Pub. Date: Apr. 26, 2007

(54) HUMAN IMMUNODEFICIENCY VIRUS ENTRY INHIBITOR ASSAY

(76) Inventor: LINGLONG ZOU, PEARLAND, TX (US)

Correspondence Address: Cheryl Liljestrand;Tanox, Inc., 10301 Stella Link Road, Houston, TX, 77025, US

(21) Appl. No.: 10/580,703

(22) PCT Filed: Nov. 24, 2004
(86) PCT No.: PCT/US04/39445

§ 371 Date: May 25, 2006

Related U.S. Application Data
(60) Provisional application No. 60/525,342, filed on Nov. 26, 2003.

(51) Int. Cl. C12Q 1/68; C12P 21/06

(52) U.S. Cl. 435/69.1; 435/6

(57) ABSTRACT

The present invention is a method for evaluating the ability of a compound to prevent the Human Immunodeficiency Virus (“HIV”) from entering into a T cell. The method involves fusing a first cell line that mimics HIV viral particles by expressing a gp120-gp41 complex on the cell surface and that contains a Tat protein in the cytoplasm with a second cell line that mimics T cells by expressing CD4 and its co-receptors on the cell surface and that contains a Tat-inducible reporter gene expression cassette in the nucleus. The cell lines are fused alone or in the presence of a potential HIV entry inhibitor and the amount of β-galactosidase produced by the fused cells is determined. Compounds that reduce the amount of 0-galactosidase produced by the fused cells have interfered with the interaction of the gp120-gp41 complex and CD4 and its co-receptors and are therefore deemed be HIV entry inhibitors.

### BACKGROUND OF THE INVENTION

1. Field of the Invention

This invention relates generally to biological assays and particularly to methods for determining the ability of a compound to prevent the Human Immunodeficiency Virus (“HIV”) from entering into a T cell or other target cell.

2. Description of the Prior Art

Acquired immunodeficiency syndrome (“AIDS”) is principally caused by a retrovirus known as the human immunodeficiency virus type 1 (“HIV”). HIV weakens the immune system by invading the body and then infecting and depleting helper T cells. Helper T cells are essential to a healthy immune system because they control the production of antibodies by B cells, maturation of cytotoxic T lymphocytes (killer T cells), maturation and activity of macrophages and natural killer cells, and numerous other regulator and effector functions of the immune system.

Infection and depletion of helper T cells occurs through a multi-step process that requires viral attachment to the CD4 receptor of helper T cells, viral attachment to coreceptor CXCR4 or CCR5, viral fusion with the cell, viral uncoating, reverse transcription of viral RNA to form DNA, synthesis of a second strand of DNA, migration of the DNA to the helper T cell nucleus, integration of viral DNA into the helper T cell genome, transcription of the DNA to produce RNA, translation of viral RNA to produce a viral polyprotein, viral protease cleavage of the polyprotein to produce viral proteins, and assembly and budding of the viral proteins to form new virus and destroy the host cell. Different drugs and treatment methods have been designed to interfere with one or more of these steps. Typical methods used to prevent or treat the disease include the use of HIV entry inhibitors, i.e., compounds that inhibit the attachment of the virus to the helper T cell or other target cells and compounds that inhibit the fusion of the virus with the target cell.

HIV viral entry into target cells occurs in distinct steps, i.e., attachment and fusion. Attachment and fusion require the interaction of several viral and cellular proteins in distinct phases: (1) attachment of viral envelope proteins to the primary receptor CD4, (2) conformational change in the viral proteins that result in the binding to a coreceptor, and (3) exposure of viral proteins that result in the fusion of the viral and target cell membranes. Attachment and fusion are principally mediated by the viral proteins gp120 and gp41. Gp120 and gp41 form a complex that is present as a trimer on the virion surface. Gp120 is the viral protein that attaches to the primary receptor CD4 on the surface of target cells. Gp120 attachment brings the virus and cell into contact although not in sufficient contact to initiate fusion. The extracellular region of CD4 consists of 4 domains (D1, D2, D3, and D4). The HIV gp120 binding site on CD4 comprises amino acids 40 to 60 (strand C′, C″, and D) in the CD4 domain 1 (D1), a stretch analogous to the CDR2 of an immunoglobulin (Ig) V domain. After attachment of gp120 to CD4, gp120 undergoes conformational change and thus binds to a chemokine coreceptor (CCR5 or CXCR4). CCR5 is the chemokine receptor used by macrophage-tropic and certain T-cell-tropic primary HIV isolates while most T-cell tropic primary HIV isolates and T-cell line-adapted HIV strains use CXCR4. Some T-cell tropic isolates are dual tropic that can use either CCR5 or CXCR4 as a coreceptor. After the interaction between HIV gp120 and the co-receptors, HIV gp41 is exposed. Gp41 then undergoes a harpoon-like conformational change that forms an attachment to the target cell membrane and then uses a spring-like mechanism to form a triple helical, u-shaped protein structure known as the “trimer of hairpins. Forming the hairpin structure draws the virus to the cell and initiates membrane fusion. This fusion results in the viral particle entering into the target cell and subsequently infect the cell.

Attempts to prevent HIV infection by inhibiting HIV viral entry have been successful. U.S. Pat. No. 6,309,880 issued to Chang, et al. on Oct. 30, 2001 (assigned to Tanox, Inc. (Houston, Tex.)) entitled “Antibodies specific for CD4-binding domain of HIV” discloses a particular epitope located within the CD4-binding region of HIV gp120 of and antibodies specific for the epitope that can inhibit HIV infection of human cells by diverse viral strains and isolates. U.S. Pat. No. 5,871,732 issued to Burkly, et al. on Feb. 16, 1999 (assigned to Biogen, Inc. (Cambridge, Mass.)) entitled “Anti-CD4 antibody homologs useful in prophylaxis and treatment of AIDS, ARC and HIV infection” discloses anti-CD4 antibody homologs useful for preventing or treating diseases in mammals, including AIDS. U.S. Pat. Nos. 6,015,881 and 6,281,331 issued to Kang, et al. on Jan. 18, 2000 and Aug. 28, 2001, respectively, (assigned to Trimeris, Inc.) entitled “Methods and compositions for peptide synthesis” discloses peptides T-20 and related peptides useful for the treatment of HIV infections. T-20, also known as pentafuside, is a 36 amino acid peptide that prevents fusion between HIV and target cells in vitro and in vivo. T20 and its analogues are derived from the C-terminus peptide segment of HIV gp41. While there are a small number of effective HIV entry inhibitors known, there exists a need for new drugs that can be used to combat HIV infections and AIDS, including HIV entry inhibitors, and a need for new methods for identifying such entry inhibitors.

Methods for identifying HIV entry inhibitors based on cell fusion assays and reporter enzymes are known. Kimpton J, and Emerman M. “Detection of replication-competent and pseudotyped human imnmunodeficiency virus with a sensitive cell line on the basis of activation of an integrated P-galactosidase gene.” J. Virol. 66:2232-2239, 1992 discloses the use of a HeLa cell line that both expresses high levels of CD4 and contains a single integrated copy of a beta-galactosidase gene that is under the control of a truncated human immunodeficiency virus type 1 (HIV) long terminal repeat (LTR). This cell line, called CD4-LTR-β-gal, can be used to determine quantitatively the titer of laboratory-adapted HIV strains. The reference does not, however, disclose the use of the cell line to evaluate compounds to determine if they are HIV viral entry inhibitors. Ciminale, V. et al., “A bioassay for HIV based on Env-CD4 interaction.” AIDS Res. Hum. Retro. 6: 1281-1287, 1990 discloses a method that uses two cell lines, one that mimics the HIV by expressing multiple HIV proteins including gp120, gp41 and that expresses tat (HL2/3) and one that mimics T cells by expressing CD4 molecules on the cell surface and that contains a tat-inducible chloramphenicol acetylltransferase (CAT) gene linked to the HIV long terminal repeat (HLCD4-CAT). Co-cultivation of the two cell lines lead to cell fusion and consequently expression of CAT enzyme. This CAT expression system, however, lacks sensitivity and takes a relatively long time to complete the measurement. The assay is typically conducted in 24-well culture plates, which limits number of samples (for screening), replication per level, or concentration levels per sample (for potency comparison) to be used. Additionally, the CAT system uses radioactive materials that are a safety risk to the operator and an environmental risk that requires special handling and disposal. Practically, the procedure is difficult to be standardized by using the CAT expression system, and it may result in unreliable results when it is applied to potency comparison between samples as well as between assays conducted in different laboratories. There is, therefore, a need for new safe, convenient, and more robust methods for evaluating potential HIV entry inhibitors and efficiently identifying new HIV entry inhibitors that may prove useful for preventing and treating HIV infections and AIDS.

### SUMMARY OF THE INVENTION

It is, therefore, an object of the invention to provide a method for determining the ability of a compound to prevent the Human Immunodeficiency Virus (“HIV”) from entering into a T cell or other target cell.

It is another object of the invention to provide a method for determining if two or more HIV entry inhibitors are synergistic when acting together to prevent the HIV from entering into a T cell or other target cell.

These and other objects are achieved by (1) providing a first cell line that mimics HIV viral particles by expressing a gp120-gp41 complex on the cell surface and that contains a Tat protein in the cytoplasm; (2) providing a second cell line that mimics T cells by expressing CD4 and its co-receptors on the cell surface and that contains a Tat-inducible reporter gene expression cassette in the nucleus; (3) co-cultivating the first cell line and second cell line using conditions that promote cell fusion; (4) measuring the amount of β-galactosidase produced by the fused cells; (5) co-cultivating the first cell line and second cell line in the presence of one or more potential HIV entry inhibitors using conditions that promote cell fusion; (6) measuring the amount of β-galactosidase produced by the fused cells; and (7) comparing the amount of β-galactosidase produced in steps (4) and (6) to determine if the potential HIV entry inhibitor is a HIV entry inhibitor, wherein the amount of β-galactosidase produced will be less in step (6) than in step (4) if the potential HIV entry inhibitor is a HIV entry inhibitor.

Other and further objects, features, and advantages of the present invention will be readily apparent to those skilled in the art.

### DETAILED DESCRIPTION OF THE INVENTION

### Definitions

The term “target cell(s)” means any cell expressing CD4 and any co-receptors such as CCR5 or CXCR4 on the cell membrane that HIV can attach and infect, e.g., helper T cells and macrophages, or any cell that can be infected by fusion between non-infected cells and HIV infected cells expressing HIV gp120 and gp41 on the cell membrane.

The term “synergism” means a cooperative effect between individual compounds such as HIV entry inhibitors such that the total effect is greater than the sum of the effects of the compounds taken independently.

This invention is not limited to the particular methodology, protocols, and reagents described herein because they may vary. Further, the terminology used herein is for the purpose of describing particular embodiments only and is not intended to limit the scope of the present invention. As used herein and in the appended claims, the singular forms “a,” “an,” and “the” include plural reference unless the context clearly dictates otherwise, e.g., reference to “a host cell” includes a plurality of such host cells.

Unless defined otherwise, all technical and scientific terms and any acronyms used herein have the same meanings as commonly understood by one of ordinary skill in the art in the field of the invention. Although any methods and materials similar or equivalent to those described herein can be used in the practice of the present invention, the preferred methods, devices, and materials are described herein.

All patents and publications mentioned herein are incorporated herein by reference to the extent allowed by law for the purpose of describing and disclosing the antibodies, polypeptides, peptides and methodologies reported therein that might be used with the present invention. However, nothing herein is to be construed as an admission that the invention is not entitled to antedate such disclosure by virtue of prior invention.

### The Invention

In one aspect, the present invention provides a method for determining the ability of a compound to prevent the Human Immunodeficiency Virus (“HIV”) from entering into a T cell or other target cell. The method comprises (1) providing a first cell line that mimics HIV viral particles by expressing a gp120-gp41 complex on the cell surface and that contains a Tat protein in the cytoplasm; (2) providing a second cell line that mimics T cells by expressing CD4 and its co-receptors on the cell surface and that contains a Tat-inducible reporter gene expression cassette in the nucleus; (3) co-cultivating the first cell line and second cell line using conditions that promote cell fusion; (4) measuring the amount of β-galactosidase produced by the fused cells; (5) co-cultivating the first cell line and second cell line in the presence of one or more potential HIV entry inhibitors using conditions that promote cell fusion; (6) measuring the amount of β-galactosidase produced by the fused cells; and (7) comparing the amount of β-galactosidase produced in steps (4) and (6) to determine if the potential HIV entry inhibitor is a HIV entry inhibitor, wherein the amount of β-galactosidase produced will be less in step (6) than in step (4) if the potential HIV entry inhibitor is a HIV entry inhibitor.

Co-cultivation of the first and second cell lines results in cell fusion. After fusion, the Tat protein in the first cell line activates the reporter gene expression cassette in the second cell line and produces the enzyme β-galactosidase. The amount of β-galactosidase produced is proportional to the amount of cell fusion that occurred.

Co-cultivation of the first and second cell lines with and without a potential entry inhibitor and a comparison of the amounts of β-galactosidase produced permits a determination of whether the potential entry inhibitor is an entry inhibitor. If the amount of β-galactosidase produced in the presence of the potential entry inhibitor is less than the amount of β-galactosidase produced in the absence of the potential entry inhibitor, the potential entry inhibitor being assayed is determined to be a HIV entry inhibitor. The potential entry inhibitor has prevented or reduced cell fusion by interfering with the interaction between the gp120-gp41 complex and the CD4 receptor and is therefore a HIV entry inhibitor. The method can be practiced using one HIV entry inhibitor or a combination of two or more entry inhibitors, e.g. a combination to two potential entry inhibitors.

The first cell line can be any cell line that expresses a gp120-gp41 complex on the cell surface and that contains a Tat protein in the cytoplasm. Preferably, the first cell line is a HL2/3 cell line that expresses a gp120-gp41 complex on the cell surface and that contains a Tat protein in the cytoplasm.

The second cell line can be any cell line that expresses CD4 and its co-receptors on the cell surface and that contains a Tat-inducible reporter gene expression cassette in the nucleus. Preferably, the second cell line is selected from the group consisting of a HeLa-CD4-LTR-β-gal cell line and a U373-MAGI-CXCR4 cell line (Vodicka Mass., et al. Indicator cell lines for detection of primary strains of human and simian immunodeficiency viruses. Virology 233: 193-198, 1997) that express CD4 and its co-receptors on the cell surface and that contain a Tat-inducible reporter gene expression cassette in the nucleus. Most preferably, the second cell line is a HeLa-CD4LTR-β-gal cell line that expresses CD4 and its co-receptors on the cell surface and that contains a Tat-inducible reporter gene expression cassette in the nucleus.

The cell lines used in the present invention are cell lines that fuse when co-cultivated under the proper conditions. Generally, the cell lines are seeded at the optimal ratio for HeLa-CD4-LTR-β-gal to HL2/3 cell line. HeLa-CD4-LTR-β-gal is seeded first, followed in turn by addition of potential HIV entry inhibitors, and seeding of HL2/3 cells. The number of total cell is maintained at 50000-75000 cells per well of 96-well culture plates. Dulbecco modified Eagle medium supplemented with 10% fetal bovine serum is used as co-cultivation medium; no antibiotics is included. The cells are cultured in a humidified 5% CO₂ incubator for at least 20 hours to allow sufficient fusion.

The Tat protein and the Tat-inducible reporter gene expression cassette are well known to skilled artisans, e.g., Bohan C A, Kashanchi F, Ensoli B, Buonaguro L, Boris-Lawrie K A, Brady J N. Analysis of Tat transactivation of human immunodeficiency virus transcription in vitro. Gene Expr. 2(4):391-407, 1992 and Negrini M, Rimessi P, Sabbioni S, Caputo A, Balboni P G, Gualandri R, Manservigi R, Grossi M P, Barbanti-Brodano G. High expression of exogenous cDNAs directed by HIV-1 long terminal repeat in human cells constitutively producing HIV-1 tat and adenovirus E1A/E1B. Biotechniques. 10(3): 344-353, 1991. Generally, Tat is a small nuclear protein of defined sequence composed of 86 or 101 amino acids depending on the viral strain. HXB2, the most popular laboratory-adapted HIV-1 strain, has a truncated Tat of 86 amino acid, and its sequence has been reported, i.e., Ratner L, Fisher A, Jagodzinski L L, Mitsuya H, Liou R S, Gallo R C, Wong-Staal F. Complete nucleotide sequences of functional clones of the AIDS virus. AIDS Res Hum Retroviruses. 3(1): 57-69, 1987. Tat-inducible reporter gene expression cassette contains HIV-1 LTR promoter (minimal sequences from −138 to +80 of LTR fragment to retain promoter activity and Tat binding site) upstream of a reporter gene sequence, followed by the poly(A) signal from and the enhancer region of simian virus 40.

The β-galactosidase produced from the cell fusion can be measured by any means known to skilled artisans. Preferably, the β-galactosidase is recovered by cell lysis after co-cultivation and measured by a sensitive chemiluminescent method known to skilled artisans, e.g., Beale E G, Deeb E A, Handley R S, Akhavan-Tafti H, Schaap A P. A rapid and simple chemiluminescent assay for Escherichia coli beta-galactosidase. Biotechniques. 12(3): 320-323, 1992; Van Poucke S O, Nelis H J. Development of a sensitive chemiluminometric assay for the detection of beta-galactosidase in permeabilized coliform bacteria and comparison with fluorometry and colorimetry. Appl Environ Microbiol. 61(12): 4505-4509, 1995; and Bronstein I, Martin C S, Fortin J J, Olesen C E, Voyta J C. Chemiluminescence: sensitive detection technology for reporter gene assays. Clin Chem. 42(9): 1542-1546, 1996. Generally, the method is based on use of 1,2-dioxetane substrate, which is commercially available. By using the substrates obtained from Applied Biosystems, there are two steps for chemiluminescently determining β-galactosidase activity. In the first step, the substrate becomes deglycosylated by the enzymatic activity of the β-galactosidase and forms a stable intermediate that accumulates during the reaction. In the second step, light emission reagent is added. This reagent shifts the pH to a value of about 10 or higher and triggers the accumulated intermediate to deprotonate and decompose with strong light emission. This chemiluminescence is measured by a luminescence plate reader. The levels of β-galactosidase produced in the various steps are compared by evaluating the chemiluminescence from each step.

The method of the present invention is useful for quickly, easily, safely, and economically evaluating a compound to determine if it is a HIV entry inhibitor. The method takes advantage of sensitive chemiluminescence detection technology and eliminates the use of radioactive materials-based technology. Also, fewer cells are required to generate measurable signal when compared to prior art methods. This means that the method can use a 96-well format instead of a 24-well format. This facilitates the analysis of several concentrations of analyte within one assay plate. Further, the 96-well format renders the assay ready for automated high throughput application. The prior art methods use expensive and dangerous radioactive components. In the present method, the use of a non-radioactive endpoint is safer for the operator and eliminates the need to dispose of radioactive materials.

In another embodiment, the invention provides a method for determining if two or more HIV entry inhibitors are synergistic when acting together to prevent the HIV from entering into a T cell or other target cell. The method comprises (1) providing a first cell line that mimics HIV viral particles by expressing a gp120-gp41 complex on the cell surface and that contains a Tat protein in the cytoplasm; (2) providing a second cell line that mimics T cells by expressing CD4 and its co-receptors on the cell surface and that contains a Tat-inducible reporter gene expression cassette in the nucleus; (3) co-cultivating the first cell line and second cell line using conditions that promote cell fusion; (4) measuring the amount of β-galactosidase produced by the fused cells; (5) co-cultivating the first cell line and second cell line using conditions that promote cell fusion in the presence of a first entry inhibitor; (6) measuring the amount of β-galactosidase produced by the fused cells; (7) co-cultivating the first cell line and second cell line using conditions' that promote cell fusion in the presence of a second entry inhibitor; (8) measuring the amount of β-galactosidase produced by the fused cells; (9) co-cultivating the first cell line and second cell line in the presence of the first entry inhibitor and the second entry inhibitor using conditions that promote cell fusion; (10) measuring the amount of P-galactosidase produced by the fused cells; and (11) comparing the amount of β-galactosidase produced in steps (4), (6), (8), and (10) to determine if the first and second entry inhibitors are synergistic, wherein the amount of β-galactosidase produced in step 4 minus step 10 is greater than the amount of β-galactosidase produced in the sum of step 4 minus step 6 and step 4 minus step 8, i.e., (step 4−step 10)>((step 4−step 6)+(step 4−step 8)). Compounds that interact to produce a synergistic effect are often advantageous because they require less compound to achieve the desired therapeutic result and cause less adverse side effects because of the lower dosage.

The potential entry inhibitors to be tested in the methods of the present invention can be any compound, drug, protein, antibody, or other molecule suspected of being a HIV entry inhibitor. For example, the potential entry inhibitors can be antibodies, antibody fragments, small organic molecules, peptides, peptide fragments, or CD4 antagonists. Such inhibitors can also be compounds such as the known HIV entry inhibitors T-1249, T-649, 5-Helix, pentafuside, and analogous compounds, PRO542, SCH-C, and AMD3100.

In another aspect, the invention provides an article of manufacture in the form of a kit comprising in separate containers in a single package (1) a first cell line that mimics HIV viral particles by expressing a gp120-gp41 complex on the cell surface and that contains a Tat protein in the cytoplasm and (2) a second cell line that mimics T cells by expressing CD4 and its co-receptors on the cell surface and that contains a Tat-inducible reporter gene expression cassette in the nucleus. The two cell lines can be used in the method of the present invention by fusing the cell in the presence or in the absence of potential HIV entry inhibitors and measuring the amount of β-galactosidase produced by such fusions using techniques and equipment described herein or well known to skilled artisans. Preferably, the first cell line is a HL2/3 cell line that expresses a gp120-gp41 complex on the cell surface and that contains a Tat protein in the cytoplasm and the second cell line is a HeLa-CD4-LTR-β-gal (HL CD4) cell line that expresses CD4 and its co-receptors on the cell surface and that contains a Tat-inducible reporter gene expression cassette in the nucleus. The kit may contain other items that make the method convenient and useful, e.g., reagents, buffers, well plates, and the like. Preferably, the kit contains a cell lysis buffer to facilitate the method, a substrate for β-galactosidase chemiluminescence determination, or a light emission enhancement solution.

### EXAMPLES

This invention can be further illustrated by the following examples of preferred embodiments thereof, although it will be understood that these examples are included merely for purposes of illustration and are not intended to limit the scope of the invention unless otherwise specifically indicated.

### Materials and Methods

### Equipment

Forma Scientific Fume Hood; Steri-Cult 200 CO₂ Incubator; Fisher Scientific Refrigerator/Freezer Combo; Fisher Scientific Isotemp 215 Water Bath; Fisher Scientific Microplate Shaker; Eppendorf 5810 R Centrifuge; Calibrated adjustable pipettes (Rannin P20, P200 and P1000); Calibrated 12-channel pipettes (Thermo Labsystems, 5-50 μl); Calibrated 12-channel pipettes (BrandTech, 20-200 μl); Tropix TR717 luminescence microplate reader; and Costar® 96-well clear-bottom opaque-well cell culture plate (Fisher Scientific, Cat: 07-200-566).

### Reagents and Buffers

Galacton-plus® β-galactosidase chemiluminescent substrate (Applied BioSystem, Cat: T2119); Light emission accelerator-II (Applied BioSystem, Cat: T2222); Dulbecco's modified eagle medium (DMEM) (Invitrogen, Cat: 10569-010); Fetal bovine serum (FBS) (Gemini, Cat: 100-106); Cell dissociation buffer (CDB) (Invitrogen, Cat: 13151-014). Lysis buffer (5x, Roche, Cat: 1897675); and Protease inhibitor pellet (Roche, Cat: 1697498). Chemiluminescence substrate reaction buffer: 100 mM phosphate buffer (PB, pH 8.) containing 1 mM of MgCl₂. To make 100 mM phosphate buffer, prepare 100 mM Na₂HPO₄ and 100 mM NaH₂PO₄ and mix them. Add 406.7 mg of MgCl₂.6H₂O to 2000 ml of PB.

### TNX-355 Reference Standard

TNX-355 Reference Standard (LN: T662-61) is a stock solution in phosphate buffered saline (PBS) containing 0.02% Tween 20 at a concentration of 4.94 mg/ml determined by OD280. The stock solution was stored at 4-8° C.

### Cell Lines

### HL2/3

HL2/3 cell line was from NIH AIDS Research & Reference Reagent Program through licensing the material for research use (License# L-006-2003/0). The cell line was obtained at passage 8. Routine culturing has been conducted in our laboratory with its propagation medium (DMEM containing 10% FBS and 0.5 mg/ml G418).

### HeLa-CD4-LTR-β-gal

The cell line was obtained from NIH AIDS Research & Reference Reagent Program. The cell line is distributed at passage 2. Routine culturing has been conducted in our laboratory with its propagation medium (DMEM supplemented with 10% FBS, 0.2 mg/ml G418, and 0.1 mg/ml hygromycin B).

### Assay Protocol

### Prepare Cells and Potential HIV Entry Inhibitors

Depending on the culture vessel (T75 or T175), 0.5−2.0×10⁶ HL2/3 or HeLa-CD4-LTR-β-gal cells were seeded into their respective Propagation Medium (for HL2/3, DMEM containing 10% FBS and 0.5 mg/ml G418; for HeLa-CD4-LTR-β-gal, DMEM containing 10% FBS, 0.2 mg/ml G418, and 0.1 mg/ml hygromycin B). Culture at 37° C. with 95% air/5% CO₂ until the cultures reach 60% confluency, but can not exceed 95% confluency.

Remove medium by aspiration with 2-ml of glass pipette. Add 5 ml (for T75 flask) or 10 ml (for T175 flask) of the cell dissociation buffer (CDB). Incubate at room temperature for at least 20 minutes. While waiting, prepare potential entry inhibitor (e.g. TNX-355) with cold Assay Medium (DMEM supplemented with 10% FBS) for treatment. Harvest cells in a 15-ml (for T75) or 50-ml (for T175) conical centrifuge tube by repeated pipetting. Determine cell density and viability in 100 μl of samples with Trypan Blue Exclusion method. Centrifuge at 1000 rpm (or 201×g) for 5 minutes. Discard supernatant and resuspend cells in pre-warmed Assay Medium at a cell density of 1×10⁶ cells/ml (for HeLa-CD4-LTR-β-gal cells) or 2×10⁶ cells/ml (for HL2/3 cells).

### Co-Cultivate Two Cell Lines

Further dilute HeLa-CD4-LTR-β-gal cells to 0.25×10⁶ cells/ml with pre-warmed Assay Medium, and pipette 90 μl to each well of a 96-well clear-bottom white-wall plate using a 12-channel pipet. Add 20 μl of drug or medium to each well as needed. Further dilute HL2/3 cells to 0.5×10⁶ cells/ml with pre-warmed Assay Medium, and pipette 90 μl to each well of a 96-well clear-bottom white-wall plate using a 12-channel pipet. Total cells in each well is 6.75×10⁴ cells at a ratio of 2:1 (HL2/3 to HeLa-CD4-LTR-β-gal cells). Culture cells at 37° C. with 95% air/5% CO₂ for about 24 hours.

### Lyse Cells

Remove the medium row by row, immediately followed by addition of 50 μl/well of Lysis Solution (Roche Lysis Buffer containing protease inhibitors). Incubate for 60 minutes at 15-25° C. with mild shaking (200-300 rpm). While waiting, dilute 1 volume of concentrated substrate with 99 volumes of Reaction Buffer to make working substrate solution.

### Chemiluminescent Reaction and Measurement

Add 100 μl of the substrate reagent to each well row by row using 12-channel pipette. Incubate for 90 minutes at room temperature with mild shaking (200-300 rpm). While waiting, bring proper volume of Accelerator II solution to room temperature. Add 50 μl of the Accelerator II to each well row by row using 12-channel pipette. Measure light emission with a microplate luminometer. The chemiluminescence readout is reported as light units per second.

### Example 1

The inhibition of HIV entry inhibitors, including TNX-355, T20, SIM.4, and sCD4, was tested on the co-cultivation-induced fusion between the two cell lines (HL2/3 to HeLa-CD4-LTR-β-gal cells). TNX-355 is a humanized anti-CD4 domain 2 antibody, T20 a synthetic oligopeptide mimicking the gp120 HR-2 region, SIM.4 a monoclonal anti-CD4 domain 1 antibody, and sCD4 is a soluble extracellular portion of CD4 molecule. Multiple levels of each reagent were tested following the procedures described in the Materials and Methods. The results are shown in Table 1.

TABLE 1
Inhibition of Cell Fusion by Various HIV Entry Inhibitors

| | |
|---|---|
| Reagent and | Response |

| | | |
|---|---|---|
| Concentration | Chemiluminescence | Inhibition (%) |

| | | | |
|---|---|---|---|
| TNX-355 | 10000 | 4978 ± 669.32 | 100.0% |
| (ng/ml) | 1000 | 6896 ± 383.09 | 95.7% |
| | 200 | 12312 ± 1516.60 | 83.5% |
| | 100 | 18724 ± 989.73 | 69.2% |
| | 50 | 23993 ± 650.74 | 57.3% |
| | 25 | 29152 ± 2492.87 | 45.8% |
| | 10 | 34525 ± 2281.39 | 33.7% |
| | 0.1 | 39185 ± 1260.79 | 23.2% |
| T20 | 10000 | 5424 ± 173.92 | 99.0% |
| (nM) | 1000 | 6314 ± 110.76 | 97.0% |
| | 200 | 7782 ± 392.30 | 93.7% |
| | 100 | 9745 ± 1097.60 | 89.3% |
| | 50 | 12813 ± 521.62 | 82.4% |
| | 25 | 18129 ± 1059.19 | 70.5% |
| | 10 | 29686 ± 2450.97 | 44.6% |
| | 0.1 | 49540 ± 1511.60 | 0.0% |
| SIM.4 | 500 | 11759 ± 518.62 | 84.8% |
| (ng/ml) | 100 | 23139 ± 1469.24 | 59.3% |
| | 20 | 34058 ± 547.37 | 34.7% |
| | 4 | 43034 ± 1209.65 | 14.6% |
| sCD4 | 1000 | 16715 ± 725.71 | 73.7% |
| (ng/ml) | 200 | 31682 ± 1067.98 | 40.1% |
| | 40 | 43286 ± 952.97 | 14.0% |
| | 8 | 43842 ± 5978.31 | 12.8% |

Referring to Table 1, the data show dose-dependent inhibition with all the known entry inhibitors tested. The results show that the method of the present invention can be used to test various compounds and determine if they are HIV entry inhibitors.

### Example 2

Assay specificity was addressed by comparing the inhibitory activity of TNX-355 to those of two structurally similar but functionally irrelevant antibodies. These antibodies are TNX-224 (a humanized antibody IgG4 which differs in complementary determining regions (CDR) but share a majority of sequence homology to TNX-355) and TNX-901 (a humanized antibody IgG1). The results are shown in Table 2.

TABLE 2
Specificity of Cell-based Bioactivity Assay for TNX-355

| Num- | | | Amount | | |
|---|---|---|---|---|---|
| ber | Antibody | Subclass | (μg/ml) | Chemiluminescence | Inhibition |
| 1 | Medium | N/A | N/A | 42728 ± 2960.35 | 0% |
| 2 | TNX-355 | IgG4 | 1.0 | 5135 ± 432.41 | 88% |
| 3 | TNX-355 | IgG4 | 0.025 | 34121 ± 714.43 | 20% |
| 4 | TNX-224 | IgG4 | 10 | 42766 ± 1605.86 | 0% |
| 5 | TNX-224 | IgG4 | 0.025 | 43489 ± 2435.93 | 0% |
| 6 | TNX-901 | IgG1 | 10 | 42847 ± 1429.78 | 0% |
| 7 | TNX-901 | IgG1 | 0.025 | 42629 ± 664.10 | 0% |

Referring to Table 2, the data show no inhibition on cell fusion when treating the cell cultures with the two functionally irrelevant antibodies even at 5- to 25-fold higher concentrations. The results show that the method of the present invention is specific to HIV entry inhibitors such as TNX-355.

In the specification, there have been disclosed typical preferred embodiments of the invention and, although specific terms are employed, they are used in a generic and descriptive sense only and not for purposes of limitation, the scope of the invention being set forth in the following claims. Obviously many modifications and variations of the present invention are possible in light of the above teachings. It is therefore to be understood that within the scope of the appended claims the invention may be practiced otherwise than as specifically described.

1-20. (canceled)

21. A method for assaying a compound or combination of compounds capable of inhibiting Human Immunodeficiency Virus (“HIV”) entry into a cell comprising:
a. co-cultivating a first cell line expressing a gp120-gp41 complex on its cell surface and comprising an inducer, and a second cell line expressing CD4 and its co-receptors on its cell surface and comprising a reporter gene, the expression of which is induced by the inducer of the first cell line, in the presence of a compound or combination of compounds under conditions that allow cell fusion;
b. comparing the level of expression of the reporter gene in the presence of said compound(s) with the level of expression of the reporter gene in the absence of said compound(s).

22. The method of claim 21, wherein the inducer is a Tat protein.

23. The method of claim 21, wherein the reporter gene is beta-galactosidase.

24. The method of claim 21, wherein the first cell line is HL2/3 cell line.

25. The method of claim 21, wherein the second cell line is a HeLa-CD4-LTR-β-gal or U373-MAGI-CXCR4.

26. The method of claim 21, wherein the compound or combination of compounds to be tested is added at varying concentrations.

27. A method for assaying for a synergistic combination of compounds capable of inhibiting Human Immunodeficiency Virus (“HIV”) entry into a cell comprising:
a. co-cultivating a first cell line expressing a gp120-gp41 complex on its cell surface and comprising an inducer, and a second cell line expressing CD4 and its co-receptors on its cell surface and comprising a reporter gene, the expression of which is induced by the inducer of the first cell line,
i. in the presence of a combination of compounds;
ii. in the presence of each individual compound of the combination alone; and
iii. in the absence of any compound; and
b. comparing the level of expression of the reporter gene for each of (i), (ii), and (iii),
wherein a combination of compounds is synergistic if the level of expression in (i) is less than the additive level of expression for each compound in (ii), as compared to the level of expression in (iii).

28. The method of claim 27, wherein the inducer is a Tat protein.

29. The method of claim 27, wherein the reporter gene is beta-galactosidase.

30. The method of claim 27 wherein the first cell line is HL2/3 cell line.

31. The method of claim 27, wherein the second cell line is HeLa-CD4-LTR-β-gal or U373-MAGI-CXCR4.

32. A kit comprising:
a. a first cell line first cell line expressing a gp120-gp41 complex on its cell surface and comprising an inducer, and
b. a second cell line expressing CD4 and its co-receptors on its cell surface and comprising a reporter gene.

33. The kit of claim 32, wherein the first cell line is in a container separate from the second cell line.

34. The kit of claim 32, wherein the first cell line is HL2/3.

35. The kit of claim 32, wherein the second cell line is HeLa-CD4-LTR-β-gal or U373-MAGI-CXCR4.

36. The kit of claim 32, further comprising one or more plates comprising wells.

37. The kit of claim 32, further comprising a cell lysis buffer.

38. The kit of claim 32, further comprising a substrate for reporter gene expression detection.

39. The kit of claim 38, wherein the substrate comprises a chemiluminescent compound.

40. The kit of claim 32, further comprising a light emission enhancement solution.
